(19) United States
(12) Patent Application Publication
(10) Pub. No.: US 2023/0046820 A1
(43) Pub. Date: Feb. 16, 2023

(54) APPARATUSES AND METHODS FOR WIRELESSLY POWERED CHARGE-BALANCED ELECTRICAL STIMULATION

(71) Applicants: NANOVISION BIOSCIENCES, INC., La Jolla, CA (US); THE REGENTS OF THE UNIVERSITY OF CALIFORNIA, Oakland, CA (US)

(72) Inventors: Abraham Akinin, Union City, CA (US); Gert Cauwenberghs, San Diego, CA (US); Chul Kim, Daejeon (KR); Patrick Mercier, San Diego, CA (US); Hiren Thacker, San Diego, CA (US)

(21) Appl. No.: 17/798,554

(22) PCT Filed: Feb. 10, 2021
(86) PCT No.: PCT/US2021/017513

§ 371 Date: Aug. 9, 2022

Related U.S. Application Data
(60) Provisional application No. 62/972,639, filed on Feb. 10, 2020.

(51) Int. Cl. A61N 1/378; A61N 1/02; A61N 1/372; A61N 1/36

(52) U.S. Cl. CPC A61N 1/3787; A61N 1/025; A61N 1/37241; A61N 1/36046; A61N 1/36175; A61N 1/37276

(57) ABSTRACT

Apparatuses and methods are disclosed for efficient wireless powering of an electrical load with precise external control over pulsed voltage waveform and metering of charge delivered. The system interfaces to an inductive coil for RF power delivery from an external duty-cycled RF power transmitter, and the electrical load. The electrical load may be a photosensitive array of electrodes for an optically addressed, electrically activated retinal prosthesis. The voltage waveform to activate the load is controlled by the transmitted RF amplitude, including switching between cathodic and anodic phases of electrical stimulation. Charge delivered to the load is quantified as discharge events through a series capacitor, transmitted by backtelemetry to the receiver for continuous monitoring throughout the stimulation phases. The subject disclosure further provides for calibration of voltage amplitude and charge metering, to compensate for variable wireless link and load conditions, through additional stimulation phases with a supplementary load with known and stable characteristics.

This patent application claims priority to U.S. Provisional Patent Application Ser. No. 62/972,639, filed Feb. 10, 2020, the content of which is hereby incorporated by reference herein in its entirety into this disclosure.

### BACKGROUND OF THE SUBJECT DISCLOSURE

### Field of the Subject Disclosure

The present subject disclosure relates to systems and methods for efficient wireless powering and control of an electrical load.

### Background of the Subject Disclosure

Neurostimulators are a class of implantable medical devices which have achieved successful clinical implementation in the past several decades. In general, they provide voltage or current pulses to electrically activate tissue in order to stimulate or suppress nerve function. Among the achievements of this technology are restoring sensory function to patients with damaged hearing, reducing the severity of tremors, treating depression, and rehabilitating voluntary motion of muscles and sphincters, among others. Almost all of these devices require implantation in a miniaturized, hermetic, and biocompatible enclosure in order to fit into the limited space available in the surroundings of the brain or the target nerve tissue. Given the importance and sensitivity of these tissues, neurostimulators operate at very high power efficiency to avoid heat damage. Another requirement of neurostimulation is charged balanced stimulation. Neurostimulators provide electrical pulses to neural tissue through specialized electrodes. As current crosses the electrode-electrolyte interface, different kinds of physical and chemical processes occur. A constant unidirectional current applied on this interface may eventually cause irreversible chemical processes that destroy the electrode and generate harmful chemical compounds that result in tissue damage. This effect also occurs in stimulators that present biphasic electrical waveforms as stimulus, but with non-zero net charge. Over time, accumulated charge imbalances can lead to the aforementioned undesirable effects.

### SUMMARY OF THE SUBJECT DISCLOSURE

Of the conventional neural stimulators available today, retinal prostheses are a type that aims to restore vision to blind patients. At this moment, retinal prostheses have not had the same clinical success as other stimulators, such as cochlear stimulators have in restoring hearing to deaf patients. The retinal prosthesis strategy generally involves electrical stimulation of the remaining retinal tissue, in the case of patients with a diseased retinal photoreceptor cells, to elicit light perception. There is a direct relationship between the geometrical characteristics of retina stimulation and the perceived shape of the perceived visual image. Thus, retinal prostheses aim to provide as many channels of stimulation as possible, in order to approximate healthy vision which can perceive high resolution 2D images. This presents a problem to the requirements of implantable neurostimulators, as a conventional high channel count neurostimulator would: generate too much heat through inefficient stimulation and high data rate video transmission; require very bulky interconnect to control so many channels; and cause tissue damage and reduced electrode lifetime due to charge unbalanced stimulation. The present subject disclosure provides, among other things, a technical solution to these technical problems.

A recent approach toward reducing the number of interconnect channels while maintaining effective high resolution stimulation was to develop a dual purpose electrode and photosensor array that could be placed under the retina. This array of photo-sensors can be globally biased with a voltage pulse using only two wires, and would produce currents from each electrode proportional to the amount of incident light on each electrode/pixel. Although there are many benefits from this approach, powering and controlling this system wirelessly while minimizing wasted power, and implant size has not been fully accomplished.

Despite the advancements in retinal prostheses, there is a need for a solution to efficiently power and control optically modulated multichannel stimulating arrays with minimal interconnect and charge balanced outputs.

### BRIEF DESCRIPTION OF THE DRAWINGS

FIG. 1 shows an RF driven charge metering stimulator driving a load in an implant inductively coupled to an external duty-cycled power transmitter, according to an exemplary embodiment of the present subject disclosure.

FIG. 2 shows an RF driven charge metering stimulator, with power subsystem, signal receiver and synchronization subsystem, stimulator subsystem, and data transmitter subsystem, according to an exemplary embodiment of the present subject disclosure.

FIG. 3A shows a power subsystem, according to an exemplary embodiment of the present subject disclosure.

FIG. 3B shows regions of operation in rectifying and regulating the AC RF input into output DC voltages of the power system shown in FIG. 3A, according to an exemplary embodiment of the present subject disclosure.

FIG. 4 shows a rectifier in the power subsystem, according to an exemplary embodiment of the present subject disclosure.

FIG. 5 shows a dual supply complementary voltage limiting regulator, interfacing to the rectifier in the power subsystem, according to an exemplary embodiment of the present subject disclosure.

FIG. 6A shows low-ranging (LO) error amplifiers in the voltage limiting regulator, according to an exemplary embodiment of the present subject disclosure.

FIG. 6B shows high-ranging (HI) error amplifiers in the voltage limiting regulator, according to an exemplary embodiment of the present subject disclosure.

FIG. 7 shows a signal receiver and synchronization subsystem, and example waveforms in the generation of the Pulse and Detect Hold signals from the RF input, according to an exemplary embodiment of the present subject disclosure.

FIG. 8 shows a stimulator core subsystem which implements adiabatic voltage stimulation and charge metering, according to an exemplary embodiment of the present subject disclosure.

FIG. 9 shows a phase logic and switch driver which resets the metering capacitor in the stimulator core subsystem, according to an exemplary embodiment of the present subject disclosure.

FIG. 10 shows a principle of operation with example timing diagram of the adiabatic charge metering stimulator, according to an exemplary embodiment of the present subject disclosure.

FIG. 11 shows a regulated supply's voltage invariant current reference, according to an exemplary embodiment of the present subject disclosure.

FIG. 12 shows a complementary high-output-swing cascode bias generator, according to an exemplary embodiment of the present subject disclosure.

FIG. 13 shows a real time comparator using a folded cascode architecture, according to an exemplary embodiment of the present subject disclosure.

FIG. 14 shows an uplink data transmission through load shift keying by parallel detuning of the secondary resonator, according to an exemplary embodiment of the present subject disclosure.

FIG. 15 shows an uplink-downlink data telemetry arbitration scheme, according to an exemplary embodiment of the present subject disclosure.

FIG. 16 shows a principle of operation with example timing diagram of the adiabatic charge metering stimulator with the addition of voltage calibration phases, according to an exemplary embodiment of the present subject disclosure.

FIG. 17 shows a stimulation phase state diagram with transitions toggled by downlink telemetry events, according to an exemplary embodiment of the present subject disclosure.

### DETAILED DESCRIPTION OF THE SUBJECT DISCLOSURE

The present subject disclosure addresses the shortcomings of conventional retinal prostheses by providing novel apparatuses and methods which offset the power load to an external component. Thus, the present subject disclosure provides, among other things, a technical solution to a technical problem, as described in detail above and appreciated by one having ordinary skill in the art.

The present subject disclosure describes apparatuses and method for RF driven charge metering stimulation comprising various components interfacing to an inductive coil, electrical load, and optional calibration load including, for example: a power subsystem, signal receiver and synchronization subsystem, stimulator subsystem, and data transmitter subsystem. The power subsystem comprises a rectifier and a dual supply complementary voltage limiting regulator along with specialized supply range extending error amplifiers. The signal receiver and synchronization subsystem demodulates downlink telemetry signals and controls the internal state machine. The stimulator subsystem implements adiabatic voltage stimulation to a photosensitive or variable load while at the same time metering the delivered charge. Charging and discharging of a series capacitor is accomplished by a reset switch that responds to the charge monitoring comparator. The system saves considerable power by being able to operate at a wide range of supply voltage, thus outsourcing voltage conversion and computational functions to the external system where inefficiencies of power transmission and conversion are not compounded. All circuits including power, comparator, references and output buffers are thus designed for wide supply operation. Uplink or backtelemetry data transmission is accomplished by detuning of the internal resonator connecting a capacitor in parallel for load shift keying of discrete charge quanta events with appropriate bidirectional communication arbitration. The system also provides for additional calibration phases to a known load, to de-embed the effect of wireless link and load uncertainty and precisely monitor the receivers available voltage supply.

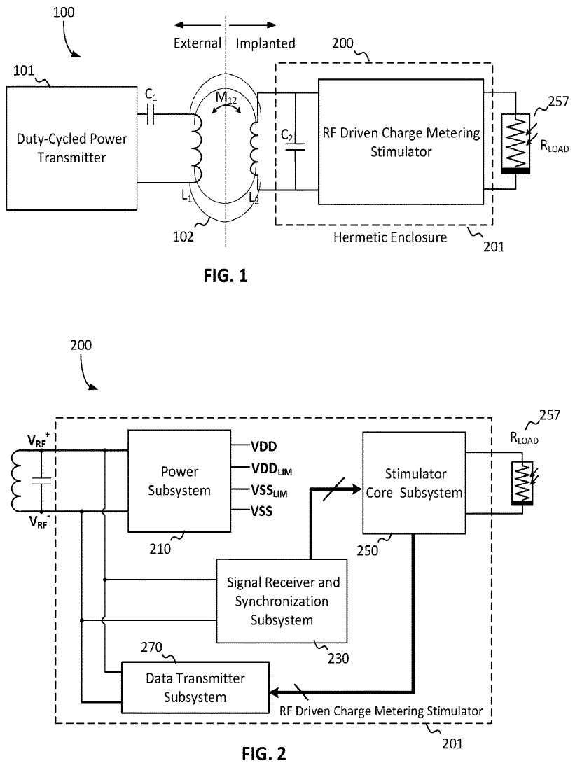

Efficiently transmitting power and control data to an inductively powered neurostimulator can be accomplished by outsourcing many of the power intensive tasks out of the implant and into the external power system where there is more space to implement energy efficient solutions, and heat from wasted power does not result in tissue damage. FIG. 1 shows an exemplary embodiment of the system concept 100, which is a wireless efficient adaptive stimulation system. A first solution is to create a system that is only powered during the time it is required to output a pulse. Therefore a duty cycled transmitter 101 is used which powers the device 200 dynamically as needed to produce pulses. This duty cycled power, is not only saving energy during the off-time of the pulse, but effectively time encoding the pulse width data into the power signal duty cycle; obviating the need to transmit, decode and process this data. The external duty cycled power supply 100 is inductively coupled 102 to the implant, which is an RF driven charge metering stimulator 200. The implant 200 may be full encased within a hermetic enclosure 201. The stimulator 200 is in turn connected to the electrode array and reference ground electrode using only two wires. To the charge metering stimulator, the electrode array is electrically equivalent to a single non-linear photosensitive load impedance, so knowledge of the output voltage is not sufficient to enforce charge balanced stimulation.

An overview of the architecture of the RF driven charge metering stimulator 200 is shown in the block diagram of FIG. 2. The system 200 designed to exemplify the implanted neurostimulator comprises of 4 major subsystems: Power 210, Data Receiver and Synchronization 230, Stimulator Core 250, and Data Transmitter 270. Each of these blocks has been designed with the principle of delegating functions to the external system 101 in order to save power. As the systems have very different functions, different strategies contribute to overall novelty and efficiency.

Power Subsystem

In order to control the amplitude of a stimulating pulse, a stimulator system can either have a variable power supply rail or make use of digital to analog converters. Power conversion in conventional neurostimulators, and many other electronic systems, usually requires the use of DC-DC converters. These converters require large capacitors, and sometimes even larger inductors to achieve high efficiency. This property makes them undesirable in miniature implants where space is a significant constraint. Additionally, DC-DC converters usually convert a fixed ratio of voltages. Alternatively, variable regulators or other digital to analog converters can generate any desired voltage level lower than a maximum constant power supply. This second approach is even more wasteful, as the system maintains a high voltage supply even as it outputs low voltage, usually completely wasting the difference in power.

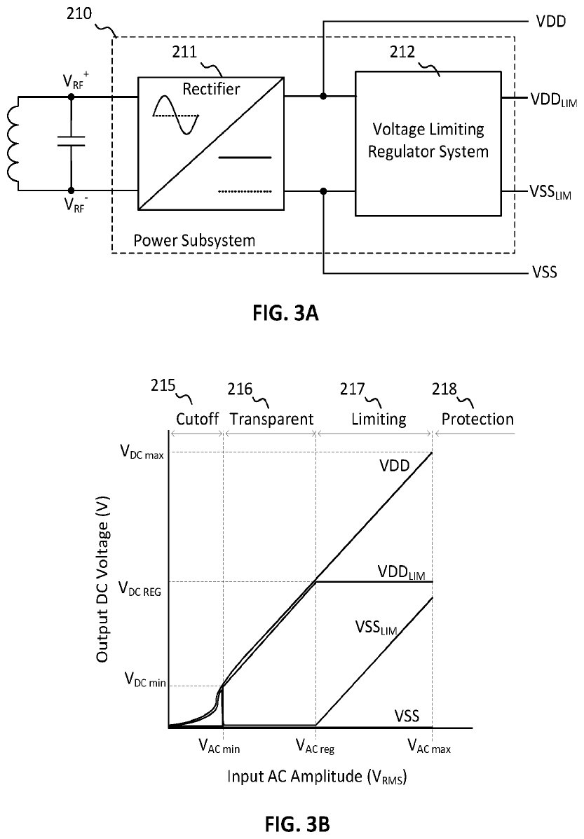

In the system of the subject disclosure, the stimulating output pulse amplitude is controlled by the external power system 101. During the duty cycled power, the implanted system 200 has an AC-DC converter, or rectifier 211, that can operate in a broad range of AC voltage amplitude. The received RF energy is rectified with low losses, and low voltage drops, to produce the system's unregulated power supply. This unregulated voltage will be directly connected to the load avoiding regulators and other intermediate steps and energy costs. By increasing or decreasing the amplitude of the external transmitter 101 we can directly control the output voltage of the stimulator 200. The cost of this energy savings is that the rectifier 211, and the rest of the system's circuits, must operate correctly at a wide range of voltage supply levels. So not only does this method save energy by avoiding voltage conversion losses, but it also saves energy by obviating the need for amplitude data transmission, detection and processing. This power distribution strategy is described in FIG. 3A.

FIG. 3A shows a power subsystem, and FIG. 3B shows regions of operation in rectifying and regulating the AC RF input into output DC voltages of the power system shown in FIG. 3A. For practical implementation of this system, not all integrated circuit processes have a wide supply range. Therefore, it is also necessary to generate low-power consuming regulated supplies in order to protect thinner gate transistors required for high speed digital and well performing analog circuits. Even though linear regulators were used to limit the analog and digital power at the high end of the RF levels, these do not significantly affect total system efficiency, as most of the power consumed by the system is taken from the unregulated supply to drive the load.

Rectifier

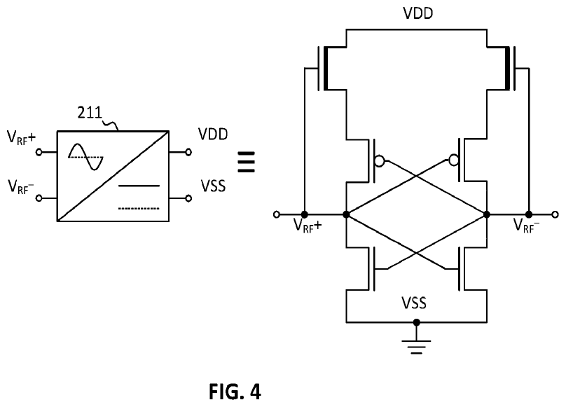

In order to accomplish the power savings and architecture simplifications that result from the aforementioned strategy, the architecture of the rectifier 211 aims to maximize power conversion efficiency and voltage conversion ratio over a wide range of input and output conditions. While there exist many architectures, they are usually optimized for a single load or voltage condition. The proposed rectifier manages very low conductive losses by a combination of fully cross-coupled complementary PMOS and NMOS pairs. Additionally, a native NMOS, or near-zero threshold device, is inserted to reduce the reverse current when (V_{RF}_{⁺}−V_{RF}_{^{−}})>0 but V_{RF}_{⁺}<VDD. FIG. 4 shows the architecture of an exemplary rectifier. This design improves on an existing method by only using one type of native transistor (n-type in this case), eliminating the redundant reverse current protection which reduces voltage drop, and making the design possible in a wider array of semiconductor processes that don't have complementary native devices. The proposed rectifier 211 also has the advantage to switch itself with the existing RF sinusoid, obviating the need for comparators, phase detectors, and phased locked loops usually present in active-rectifiers. These three mixed signal blocks require significant design effort, greatly increase power overhead, and generally must be optimized for a narrow range of operating voltage and frequency. Therefore, this rectifier presents significant improvement over previous strategies as it has low power consumption overhead, and its wide operability can enable external transmitter control of stimulation amplitude.

Dual Complementary Regulators

Many semiconductor processes provide higher-voltage-tolerant transistors as well as smaller, faster, standard transistors useful for high performance analog and digital operations. In this design, both kinds are harnessed to extend the functional range. In order to execute the power strategy proposed in FIG. 3B, we require regulation of the main power supply VDD. As the goal is to make secondary supplies that will not destroy the low-voltage devices, we require a limited voltage supply with respect to ground VSS, as well as another limited voltage with respect to VDD. In order to accomplish this we have designed a dual complementary low dropout regulator to limit both supply rails.

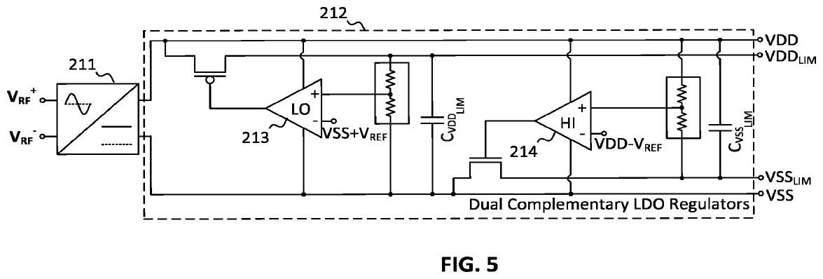

The dual regulator architecture, shown in FIG. 5, has 3 main regions of operation shown in FIG. 3: cutoff 215, transparent 216, and limiting 217. In the Cutoff region 215, the unregulated voltage VDD is too low to power the error amplifiers that control the pass transistors, thus the regulated supplies are turned off if VDD is less than V_{DC min}. In the Transparent region 216, the regulators turn on the pass transistor such that the regulated supplies are almost the same amplitude as the unregulated supplies (except for the dropout voltage across the pass transistors). In the Limiting region 217, when VDD exceeds the safe limit of the low-voltage transistors, V_{DC max LV} the error amplifiers decrease the conductivity of the NMOS and PMOS pass transistors to maintain VDD_{LIM} at V_{DC max LV} and VDD−VSS_{LIM} at V_{DC max LV}. Finally, a fourth region 218 exists where overvoltage Protection prevents breakdown of all circuits above V_{AC max}. This design improves on existing regulator architectures in that the error amplifiers, driven by the unregulated supply as a power source, default to complementary high (HI), and low (LO) voltages when the supply is insufficient to operate the amplifier correctly

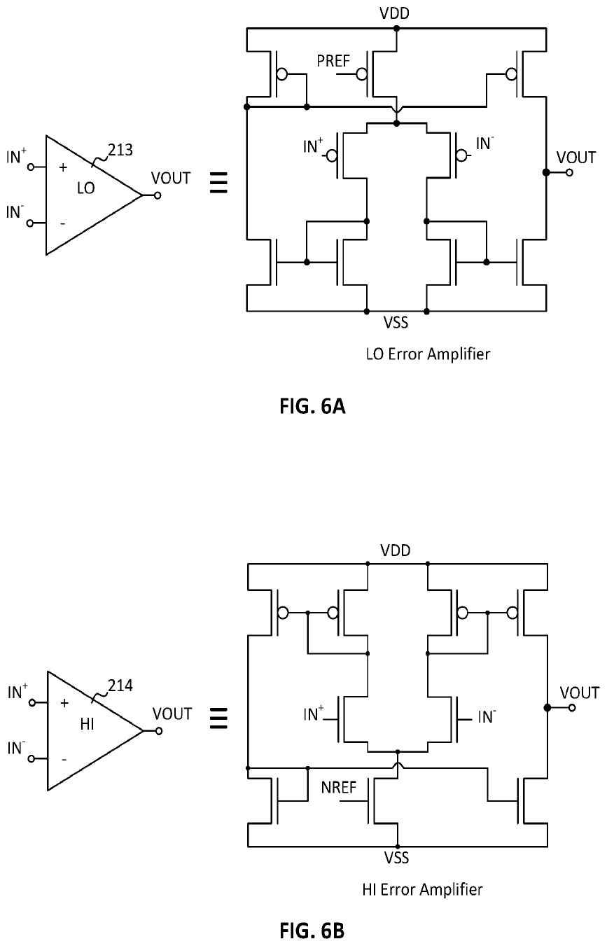

One possible architecture for the complementary error amplifiers is shown in FIGS. 6A-6B. The use of complementary-defaulting-to-rail architectures is what enables the operation of the regulator at lower VDD voltages and thus decreasing V_{DC min} and correspondingly V_{AC min}. This allows the permissible output voltage range of the stimulator to span [V_{DC min}, V_{DC max HV}], while utilizing the advantages of both high voltage and low voltage transistors.

Data Subsystem

The signal receiver and synchronization subsystem consists of a downlink telemetry receiver, clock recovery circuit, power-on reset circuit, and system state machine. Its purpose is to receive data signals from the external controller, recover a clock of the same frequency as the carrier wave, and setup the correct sequence of calibration and stimulation. As part of the strategy to reduce the amount of operations on the implantable system, the only data transmitted downlink is a single bit asynchronous, time encoded, amplitude modulated pulse signaling a change in the stimulation phase. As described previously, the amplitude of the stimulation waveform and the duration of the stimulation waveform are analog encoded on the RF signal by the external transmitter, to minimize power dissipation and operational complexity in the implant.

Data Receiver

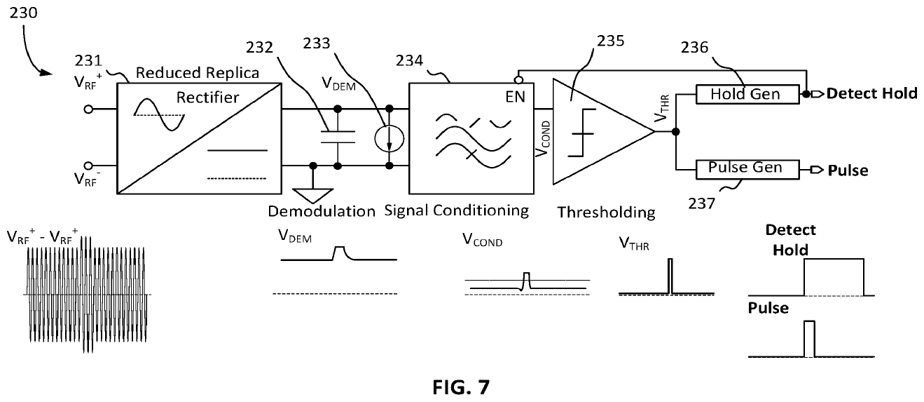

In order to receive the phase-changing data pulses the amplitude modulated RF wave is demodulated. In this system we propose a strategy for demodulation that involves the proposed rectifier 231 shown and described in FIG. 4 as rectifier 211. The system diagram of the downlink data receiver is depicted on FIG. 7. As the proposed rectifier 231 is an efficient, wide input range circuit, the same architecture can be applied toward demodulating the RF signal envelope. In order to minimize power syphoned away from the rectifier system, this auxiliary rectifier 231 is many times narrower than the primary power rectifier 211, as it needs to drive a much smaller load. After the signal demodulating rectifier 231, the signal encounters an integrating capacitor 232 and a current sink 233 that permits the demodulated voltage to decrease after the modulated signal pulse is over. Subsequently a mixed signal active bandpass filter 234 conditions the signal to enhance the pulse. After the bandpass filter 234, a real time comparator 235 detects threshold crossing pulses. When a pulse is detected, a circuit, Pulse Gen 236, generates a digital pulse signal of a standard duration, while another circuit, Hold Gen 237, generates a much longer hold signal that digitally resets the bandpass filter and prevents any duplicate events from detection for a refractory period.

Stimulator Subsystem

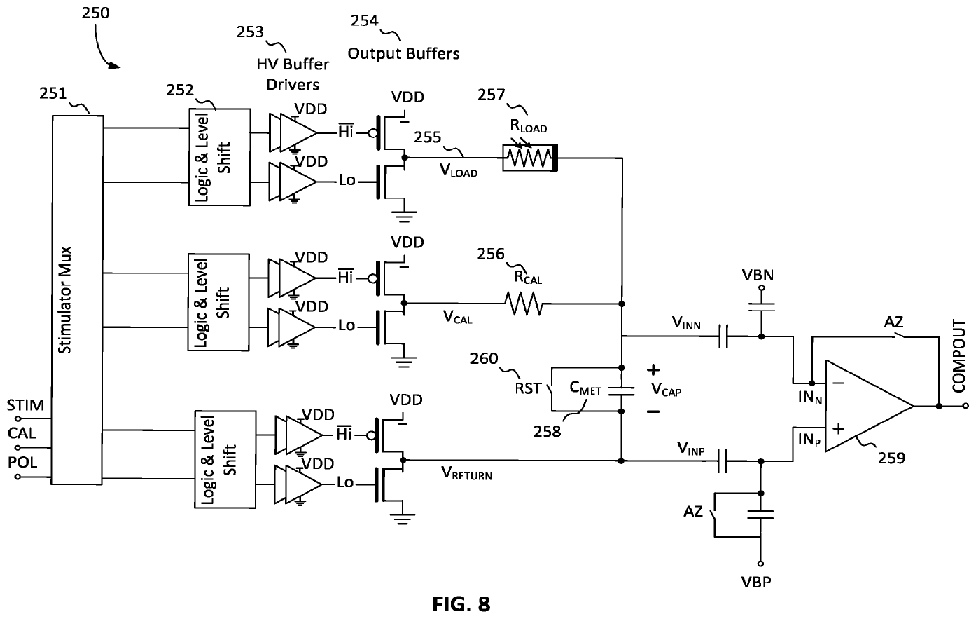

FIG. 8 shows an exemplary embodiment of the stimulator subsystem 250. The stimulator subsystem 250 provides a voltage pulse waveform by directly connecting the duty-cycled and amplitude modulated supply VDD to the desired load. To this end, the stimulator subsystem 250 relies on three tri-state switches, or Output Buffers 254, that can connect each terminal of either the intended load 255, or a known calibration resistor 256 to VDD or VSS. As the current flows through the load R_{LOAD} 257, metering capacitor C_{MET} 258 begins charging until it reaches a set differential voltage threshold, at which point the comparator 259 monitoring this voltage activates a reset switch to discharge the capacitor. Each time there is a discharge event, an amount of charge Q=C_{MET} Vₜₕᵣₑₛₕ. Therefore this stimulator 250 outputs an analog voltage of arbitrary amplitude to drive a load of unknown impedance while outputting digital counts of the delivered charge. This method conserves a lot of power, and prevents complexity and error in the system compared to a series transimpedance amplifier or series resistor current measurement. A transimpedance amplifier is impractical as currents of the order of milliamperes would need to be driven through an operational amplifier at great cost in headroom power. Similarly, a series resistor current measurement would require a precise, linear, high bandwidth amplifier, and an accurate analog to digital converter to quantize the current followed by digital integration to calculate total charge. Instead, the proposed system is not only simpler and more power efficient, but the charge quantization signals may be directly used to send backtelemetry events to the external system.

The switches in the Output Buffers 254 are designed to have very low impedance in order to reduce power consumption and voltage drop across them. They also have to be built to withstand the full range of stimulation voltage, and so in this implementation they are designed to use high voltage tolerant IO transistors. In order to have both low impedance, especially at very low voltages, and tolerate high voltages, the switches were sized considerably large in relation with the rest of the system. Although the area occupied by the switches is significant, it is an acceptable trade-off for the large range of operation of the stimulator, which is approximately [0.5-3V] in the implemented process, but may be significantly higher in processes with higher voltage tolerant devices. The output buffers 254 are preceded by HV Buffer Drivers 253, output multiplexor logic 251, and voltage level shifters 252.

Although the output buffers 254 and corresponding drivers are implemented with high voltage tolerant devices, the rest of the stimulator 250 is entirely composed of standard gate thickness low voltage devices, for size speed, and threshold voltage considerations. In order to operate in potentially breakdown inducing conditions, several strategies were taken to protect the circuits while utilizing the advantages of the standard devices.

The comparator 259 required to detect whether C_{MET} 258 has exceeded the desired threshold voltage is capacitively coupled preventing DC overvoltage. This capacitive coupling is also advantageous to apply a differential bias through VBN VBP. In this way the comparator 259 acts like an open loop difference differential amplifier. The comparator also has the capability to perform an autozero cycle to eliminate intrinsic offsets and set the otherwise-floating input voltage operating point.

Another component of the stimulator may be the reset switch RST 260. A diagram describing these switches and supporting structures is shown in FIG. 9. An exemplary phase logic metering switch driver is shown in the figure. As switching is desired to be fast and efficient, low voltage standard devices are used. In order to prevent overvoltage at the reset switch, several strategies are applied. First the NMOS reset switch is isolated through triple well, and as usual the PMOS switch is implemented in its own n-well. Second, the comparator prevents the source drain voltage from exceeding a set threshold. Third the high speed drivers required to switch the reset switches on and off are powered by a muxed power supply. For example, in the positive current phase, if VDD exceeds V_{DC max LV}, the RSTs NMOS transistor driver is driven with VDD_{LIM}. Conversely, in the reverse current phase when the switch terminal VDUTN is connected to VDD, the RSTs PMOS is driven with VSS_{LIM} as a low supply rail. To make this possible, logic circuits, a power rail selector mux, and dual rail level shifters (level translators) are implemented on the drivers. The drivers are sized for maximum speed of reset using the principles of logic effort sizing.

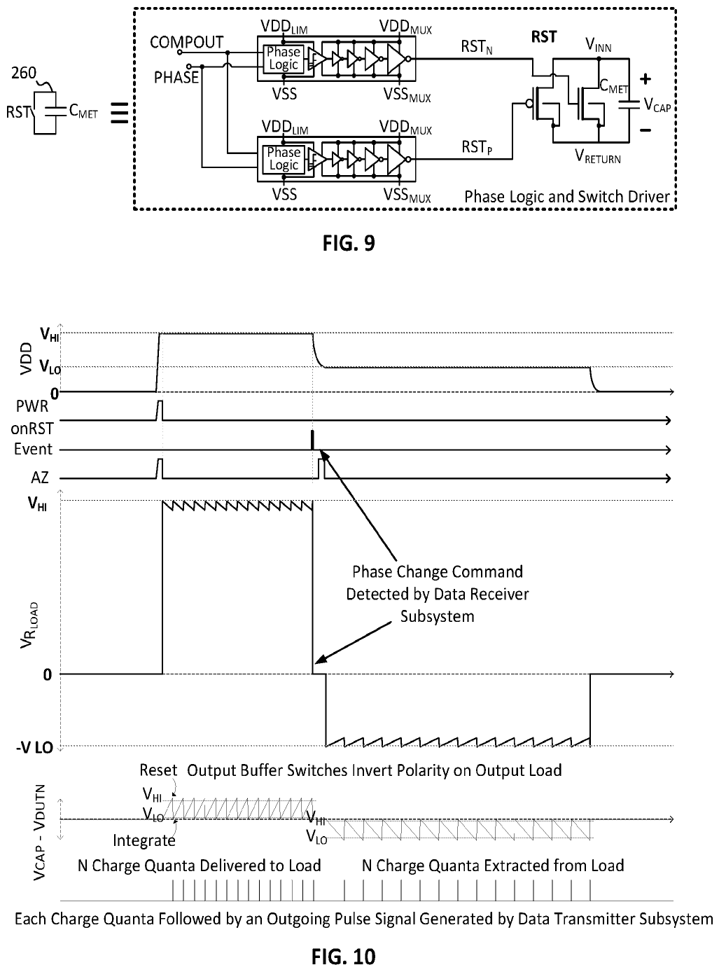

A description of the signals involved in the adiabatic charge metering stimulator are represented in FIG. 10. As the unregulated supply voltage VDD charges up to V_{HI} at the beginning of a duty cycled pulse the comparator undergoes autozero and the state machine is reset. Thereafter, VDD is connected to the load providing positive V_{HI} of voltage across the load terminals. During this time, the metering capacitor charges and discharges as the comparator input reaches threshold. The voltage excursion of the metering capacitor causes a triangle ripple voltage across the applied load voltage. However, considering it is desirable for the reset threshold to be low, and the switching frequency to be much higher than the stimulation (pulse repetition) frequency; it will not affect the performance of this system as a neurostimulator. For each charge quantum completed, there is an immediate backtelemetry event pulse. When the first phase is complete the external system sends an upmodulated pulse to signal a phase change, and the amplitude of the RF signal is immediately decreased to change VDD to V_{LO}. After a brief autozero period, the stimulator turns on applying a negative V_{LO} voltage across the same terminals mentioned above. Similarly, charge quanta cause resets, which in turn are transmitted back to the external system. Enforcing the reverse phase duration to contain the same number of charge quanta as the first, in other words charge balance, can simply be done from the external system.

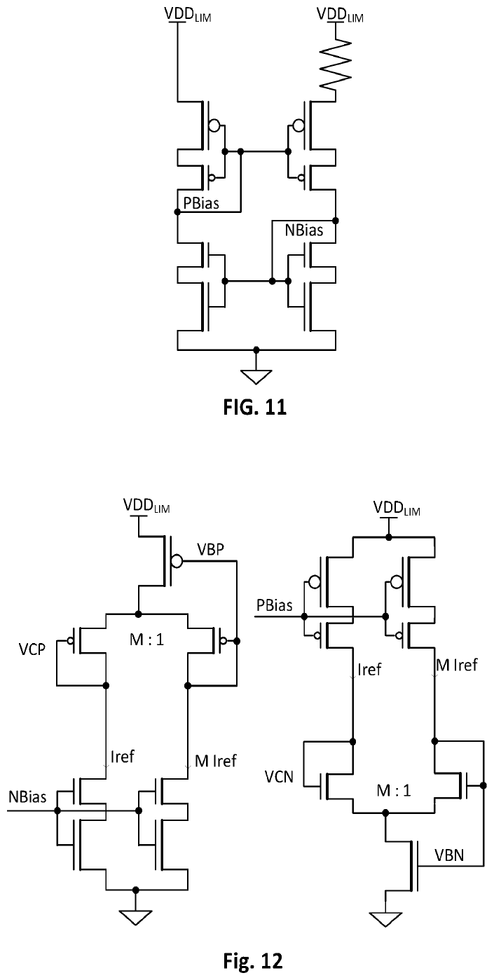

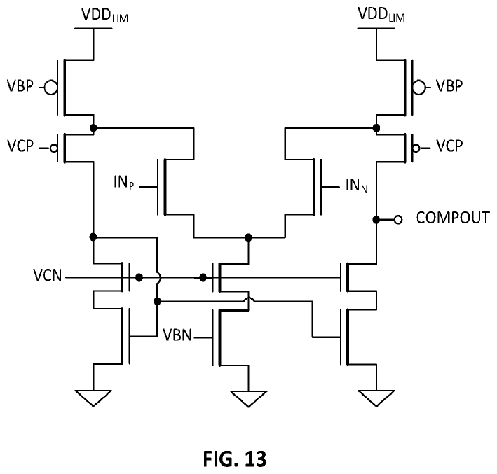

FIG. 11 shows the current reference with supply variation rejection circuit, and FIG. 12 shows high output swing bias generator circuit, for the real time comparator 259 in the stimulator 250. The comparator 259 itself is a folded cascode amplifier designed with reduced threshold devices in order to operate at very low voltage. The combination of the design choices for the current and bias generators, as well as the amplifier enable the extended operating range which is a benefit of this design. The architecture of the folded cascode comparator is shown in FIG. 13.

Data Transmitter Subsystem

Finally, the backtelemetry subsystem 270 is responsible for transmission of uplink data from the implant 200 to the external transmitter 101. Whereas downlink data was transmitted through amplitude shift keying (ASK). Uplink data is transmitted through load shift keying (LSK). The external and internal resonators in the system, described in FIG. 1, are coupled in such a way that changes in the resonance of the implanted resonator (or even an extreme and sustained rise in power consumption) can be observed on the external system as a reflected impedance.

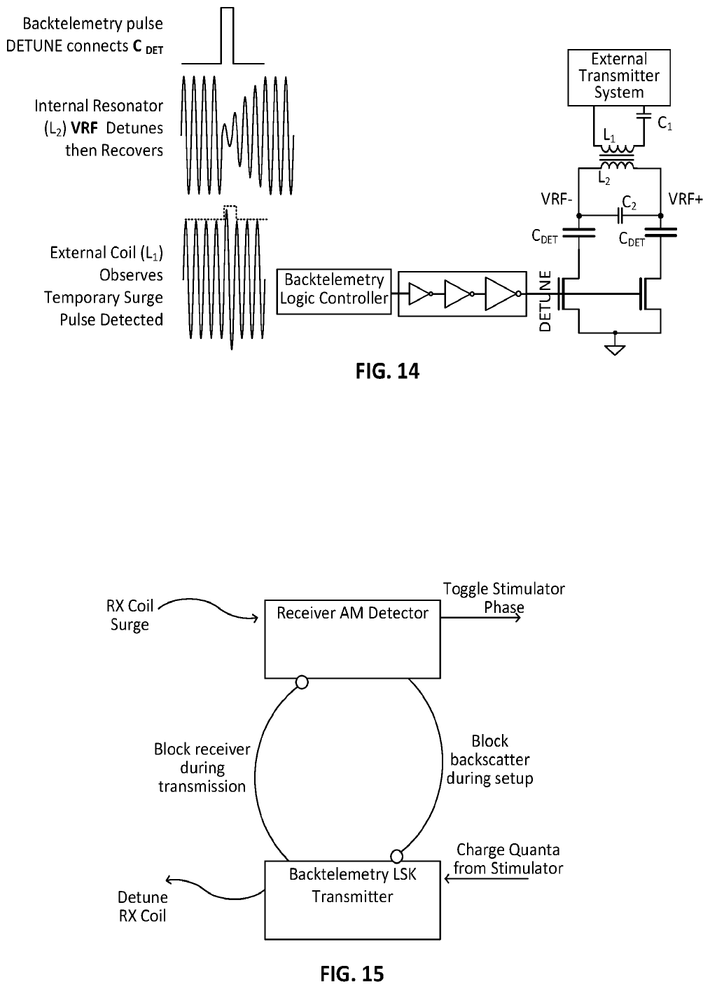

When a charge quanta has been delivered to the load, FIG. 14 shows how a driver and switch system connects additional capacitors to the resonator in order to detune the system and send a backtelemetry pulse signal. This figure shows data transmission strategy vertical. Though higher duration backtelemetry pulses and greater magnitude of detuning generate a stronger signal at the external transmitter, they also consume large amounts of power which may inadvertently power down the system

In order to prevent incoming and outgoing events from colliding, an uplink/downlink arbitration scheme at the implant is proposed and implemented as a timed state machine, shown in FIG. 15. An additional, but similar system may be included in the external system that ensures data is transmitted faithfully.

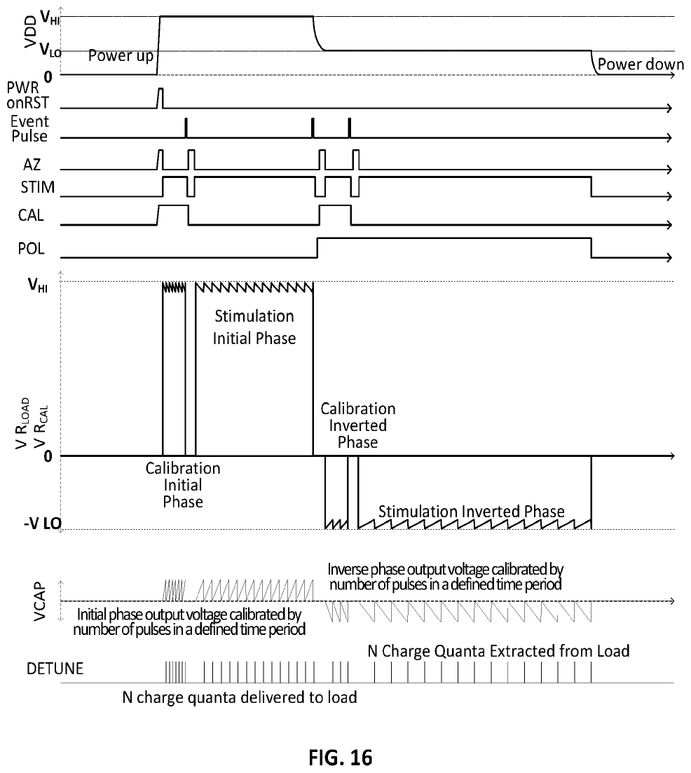

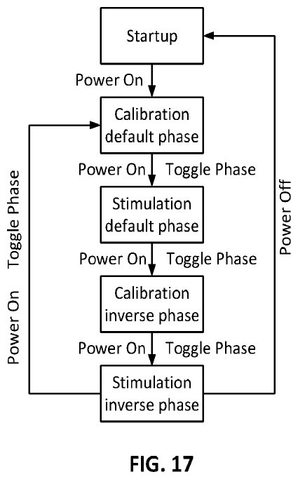

A remaining detail in the functionality of the system is worthy of attention. The external and internal systems are loosely inductively coupled. As this implementation details a retina implant application the coil may be tethered to the eyeball, changing the coupling coefficient whenever eye movements such as saccades and microsaccades occur. In order to ascertain the exact value of the implant's VDD at the time of stimulation, an additional known resistance is provided as a test load. By connecting this resistor to VDD and monitoring the number of charge metering pulses, the system transmits to the external system the information required to calculate VDD. FIG. 16 shows the role of calibration on the system and how charge quanta pulses can be used both to enforce charge balanced stimulation and closed loop voltage control. Having discussed the reasons and implementation of calibration, FIG. 17 shows the implemented state machine of the system cycling through calibration and stimulation phases as downlink phase changing events are sent from the external system.

The foregoing disclosure of the exemplary embodiments of the present subject disclosure has been presented for purposes of illustration and description. It is not intended to be exhaustive or to limit the subject disclosure to the precise forms disclosed. Many variations and modifications of the embodiments described herein will be apparent to one of ordinary skill in the art in light of the above disclosure. The scope of the subject disclosure is to be defined only by the claims appended hereto, and by their equivalents.

Further, in describing representative embodiments of the present subject disclosure, the specification may have presented the method and/or process of the present subject disclosure as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present subject disclosure should not be limited to the performance of their steps in the order written, and one skilled in the art can readily appreciate that the sequences may be varied and still remain within the spirit and scope of the present subject disclosure.

What is claimed is:

1. An apparatus, comprising:
an implant comprising an RF driven charge metering stimulator; and
a duty cycled power transmitter which powers the implant dynamically as needed to produce pulses.

2. The apparatus in claim 1, wherein the power transmitter is only powered when required to output a pulse.

3. The apparatus in claim 1, wherein the power transmitter is inductively coupled to the implant.

4. The apparatus in claim 1, wherein the stimulator is connected to an electrode array and reference ground electrode.

5. The apparatus in claim 1, wherein the stimulator includes a power subsystem.

6. The apparatus in claim 5, wherein the power subsystem includes a rectifier that can operate in a broad range of AC voltage amplitude.

7. The apparatus in claim 6, wherein the rectifier includes only one type of native transistor.

8. The apparatus in claim 6, wherein the rectifier can switch itself with existing RF sinusoid.

9. The apparatus in claim 1, wherein the stimulator includes a data subsystem.

10. The apparatus in claim 9, wherein the data subsystem comprises a downlink telemetry receiver, clock recovery circuit, power-on reset circuit, and system state machine to receive data signals, recover a clock of same frequency as carrier wave, and set up a correct sequence of calibration and stimulation.

11. The apparatus in claim 1, wherein the stimulator includes a stimulator core subsystem.

12. The apparatus in claim 11, wherein the stimulator core subsystem provides a voltage pulse waveform by directly connecting a duty cycled and amplitude modulated supply VDD to a desired load.

13. The apparatus in claim 12, wherein the stimulator core subsystem relies on three tri-state switches that can connect each terminal of either the intended load, or a known calibration resistor to VDD or VSS.

14. The apparatus in claim 13, wherein the three tri-state switches have very low impedance in order to reduce power consumption and voltage drop across them.

15. The apparatus in claim 14, wherein the three tri-state switches use high voltage tolerant IO transistors.

16. The apparatus in claim 11, wherein the stimulator core subsystem includes a reset switch.

17. The apparatus in claim 11, wherein the stimulator core subsystem includes a comparator.

18. The apparatus in claim 11, wherein the stimulator core subsystem implements adiabatic voltage stimulation to a photosensitive or variable load while at the same time metering the delivered charge.

19. The apparatus in claim 1, wherein the stimulator includes a data transmitter subsystem.

20. The apparatus in claim 19, wherein the data transmitter subsystem provides transmission of uplink data from the implant to the power transmitter.
